(19) United States
(12) Patent Application Publication
(10) Pub. No.: US 2023/0031548 A1
(43) Pub. Date: Feb. 2, 2023

(54) COMPOSITION FOR OPTIMIZED DIETARY SUPPLEMENT FORMULATIONS

(71) Applicant: REDBUD BRANDS, LLC, Huntington Beach, CA (US)

(72) Inventor: John Ferrari, Newport Beach, CA (US)

(21) Appl. No.: 17/878,984

(22) Filed: Aug. 2, 2022

Related U.S. Application Data
(60) Provisional application No. 63/228,478, filed on Aug. 2, 2021.

(51) Int. Cl. A61K 31/315; A61K 31/045; A61K 31/335; A61K 33/06; A61K 31/122; A61K 31/07; A61K 33/26

(52) U.S. Cl. CPC A61K 31/315; A61K 31/045; A61K 31/335; A61K 33/06; A61K 31/122; A61K 31/07; A61K 33/26

(57) ABSTRACT

The present invention relates to compositions for human consumption of tailored dietary supplements or nutraceuticals, comprised of various components including, among others, vitamins and minerals, provided in various permutations, in multiple daily doses, based on an individual's age and gender, bioavailability, and synergistic and competing properties of the components, thereby promoting general wellbeing and healthiness.

### CROSS-REFERENCE TO RELATED APPLICATIONS

This application claims the benefit of U.S. Provisional Application 63/228,478 filed on Aug. 2, 2021, the disclosure of which is incorporated herein by reference.

### FIELD OF THE INVENTION

The present invention relates to compositions for human consumption of tailored dietary supplements or nutraceuticals, comprised of various components including, among others, vitamins and minerals, provided in various permutations, in multiple daily doses, based on an individual's age and gender, bioavailability, and synergistic and competing properties of the components, thereby promoting general wellbeing and healthiness.

### BACKGROUND OF THE INVENTION

Dietary supplement use is common in the United States. During 2017-2018, 57.6% of U.S. adults used any dietary supplement in the past 30 days. The percentage of adults using dietary supplements increased with increasing age. Dietary supplement use was higher among women than men in all age groups. The use of multiple (two, three, and four or more) dietary supplements increased with increasing age; nearly one-quarter of adults aged 60 and over (24.9%) reported taking four or more dietary supplements. Multivitamin-mineral supplements were the most common dietary supplements used by adults in all age groups, followed by vitamin D and omega-3 fatty acid products. From 2007-2008 through 2017-2018, the percentage of adults reporting dietary supplement use increased in all age groups. A high level of dietary supplement use can contribute substantially to nutrient intake in the United States, potentially mitigating nutrient shortfalls as well as increasing the risk of excessive intake, especially with high concurrent use of more than one product.

Supplements are poorly regulated in an industry valued at over $40 billion annually. Consumers carry the burden of navigating their options and often do not have confidence or training necessary for getting it right. Four or five consumers are confused about their nutrition, and seven out of ten think the FDA is unable to keep them safe from harmful supplement products. Over 76,000 supplement products are marketed to consumers, most of which simply do not work. Many leading brands contain near-toxic levels of nutrients and combine nutrients to the point of nullifying their effects on the body. Single pill multivitamins and nutrients don't work and they take advantage of unknowing consumers. Some nutrients can undermine one another and should not be taken together, while others need to be paired with other nutrients for optimal absorption. Many multivitamins contain near-toxic and potentially dangerous amounts of a nutrient that your body can't even process—in the world of supplements, more is not better. You cannot lean solely on supplements for your nutrition—vitamin A from food is not the same as vitamin A from a supplement. Many nutrients are inhibited by coffee and/or food, which undermines the classic “morning multi”. Consumers need real experts—dietitians, the only licensed and accredited medical nutrition professional in the US to clean up the misinformation.

Among U.S. adults aged 20 and over, 57.6% used any dietary supplement in the past 30 days, and use was higher among women (63.8%) than men (50.8%); dietary supplement use increased with age, overall and in both sexes, and was highest among women aged 60 and over (80.2%); the use of two, three, and four or more dietary supplements increased with age, while the percentage of adults not using any dietary supplement decreased with age; the most common types of dietary supplements used by all age groups were multivitamin-mineral supplements, followed by vitamin D and omega-3 fatty acid supplements; from 2007-2008 through 2017-2018, the prevalence of dietary supplement use increased in all age groups among U.S. adults. The additional nutrients provided by dietary supplements can help meet recommended nutrient targets. The average American adult consumer spends $635 annually on nutritional supplements. Over two-thirds of consumers are interested in personalizing supplement and nutraceutical use with ingredients that are specific to their personal needs. Nearly eight out of ten Americans, male and female over the age of 18, report taking dietary supplements. Over 20% of consumers are considered heavy supplement users (4+ types per day), in 2015, up from 18% in 2006.

The average American diet has significant nutrient shortfalls that necessitate supplementation. It is widely known that an adequate intake of vitamins and minerals is essential for optimal health, wellness and general wellbeing. Most individuals do not consume a healthy and balanced diet necessary to meet the minimum Recommended Daily Allowance (RDA) of all nutrients. Deficiencies in the consumption of nutrients can lead to a breakdown of the structures and functions of human physiology. While there are a large variety of dietary supplements on the market, they are not designed to address the unique needs of most individuals. A wide variety of factors influence the nutritional needs of an individual, and no two individuals are necessarily are the same. Consideration should be given to a person's age and gender, the bioavailability of nutrients, interactions between certain nutrients and supplements, interactions with food, and the circumstances and time of ingestion are beneficial.

### OVERVIEW OF THE INVENTION

Provided herein are oral compositions of tailored dietary supplements or nutraceuticals for human consumption. One preferred embodiment of the present invention consist of compositions of tailored dietary supplements or nutraceuticals. Another preferred embodiment, comprised of various components including, among others, vitamins and minerals, provided in various permutations in multiple daily doses, based on an individual's age and gender, thereby promoting general wellbeing and healthiness.

One preferred embodiment of the present invention includes three supplement consumption occasions per day. In the morning, a slow-release tablet survives your morning coffee, then releases magnesium, zinc, vitamin E, manganese, and calcium into your body. In the afternoon, a tablet of copper, iron, phosphorus, molybdenum, calcium and vitamin K, taken 2-3 hours after lunch for optimal absorption. If consuming with a snack, avoid phytic acids, oxalic acids, polyphenols, egg yolk, and manganese. In the evening, a before-bedtime chewable that includes calcium, potassium, selenium, chromium, iodine, vitamin A, vitamins B1, B2, B3, B5, B6, B7 and B12 vitamin C, vitamin D, choline and methylated folate for overnight recovery.

In another preferred embodiment the afternoon and evening consumption occasions are recommended to be consumed on an empty stomach, in between meals in the afternoon and in the evening before bed.

In one preferred embodiment, the formulation includes additional instructions for consumption of the afternoon consumption occasion, if needed to be consumed with a snack to avoid upset stomach, consume with a snack excluding phytic acids, oxalic acids, polyphenols, egg yolk, and manganese.

In one preferred embodiment, thiamin destruction is prevented by the presence of reducing compounds such as Vitamin C included in the evening consumption occasion formulation.

In one preferred embodiment, Vitamin E is separated from Vitamin K and Vitamin A, because they are all inhibitors to each other.

In one preferred embodiment, manganese taken in the morning consumption occasion in a time-release capsule to avoid interactions by copper, fiber and oxalic acids in one's breakfast that may interfere with its absorption.

In one preferred embodiment, phosphorus is kept separate from magnesium and zinc to avoid inhibition of phosphorus by the same.

In one preferred embodiment, calcium and magnesium are kept to separate eating occasions because they can compete for absorption.

In one preferred embodiment, the intake of calcium in spread among consumptions occasions for an optimal absorption rate.

In one preferred embodiment, vitamin A is included in the evening consumption occasion in the form of beta-carotene and palmitate.

In one preferred embodiment, the afternoon and evening consumptions occasions to be consumed on an empty stomach, in between meals in the afternoon and in the evening before bed. Additional instructions for the afternoon consumption occasion, if needed to be consumed with a snack to avoid upset stomach, consume with a snack excluding phytic acids, oxalic acids, polyphenols, egg yolk, and manganese.

In one preferred embodiment, the morning consumption occasion is provided in a time-release capsule to avoid inhibiting interactions.

In one preferred embodiment, manganese is taken in the morning consumption occasion to avoid interactions with one's breakfast.

In one preferred embodiment, thiamin is taken in the evening consumption occasion to prevent destruction of thiamin by the presence of reducing compounds such as vitamin C.

In one preferred embodiment, chromium is taken at the evening eating occasion to be taken on an empty stomach in order to avoid inhibition by oxalates and phytic acids in food.

One preferred embodiment includes phosphorus in the afternoon consumption occasion to avoid inhibitory effects of the insoluble complexes formed between some minerals and prosperous

In one preferred embodiment, the invention offers the correct amount of 26 essential nutrients. The program contains the same 26 essential nutrients in amounts that vary depending on gender and life stage.

Embodiments can include a time release components that begin working at various durations consumption.

Embodiments can also include a coated capsule comprising a liquid fill, a shell surrounding the liquid fill, and a coating around the shell. The liquid fill may optionally comprise the vitamins and minerals, a liquid vehicle, a plasticizer, and water. The shell may comprise gelatin, an opacifier, optionally a colorant, and optionally water. The coating may comprise a cellulose derivative, a polyether, a mineral oil, and vitamins and minerals. The liquid vehicle may comprise a vegetable oil, and the plasticizer may comprise glycerol. The opacifier may comprise titanium dioxide. The cellulose derivative may comprise hypromellose and the polyether may comprise PEG 400. The liquid fill may comprise 500 mg green tea extract, 300 mg vegetable oil, 50 mg glycerol, and water. The shell may comprise 265 mg gelatin, 10 mg titanium dioxide, and 1 mg colorant. The coating may comprise 52 mg hypromellose, 28 mg PEG 400, 6 mg mineral oil, 1 mg melatonin, and 1 mg chamomile extract.

### BRIEF DESCRIPTION OF THE DRAWINGS

The accompanying drawings, which are included to provide a further understanding of the invention and are incorporated in, and constitute a part of, this specification, illustrate preferred embodiments of the invention and together with the detail description serve to explain the principles of the invention. In the drawings:

FIG. 1A is an exemplary table of morning components.

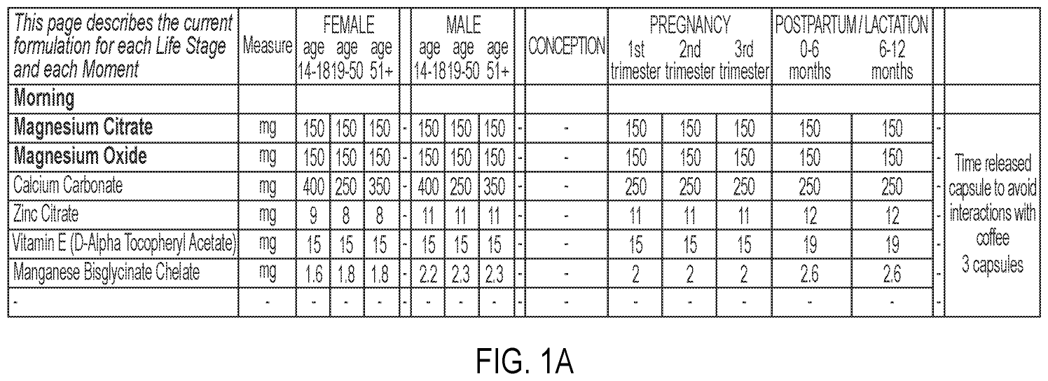

FIG. 1B is an exemplary table of afternoon components.

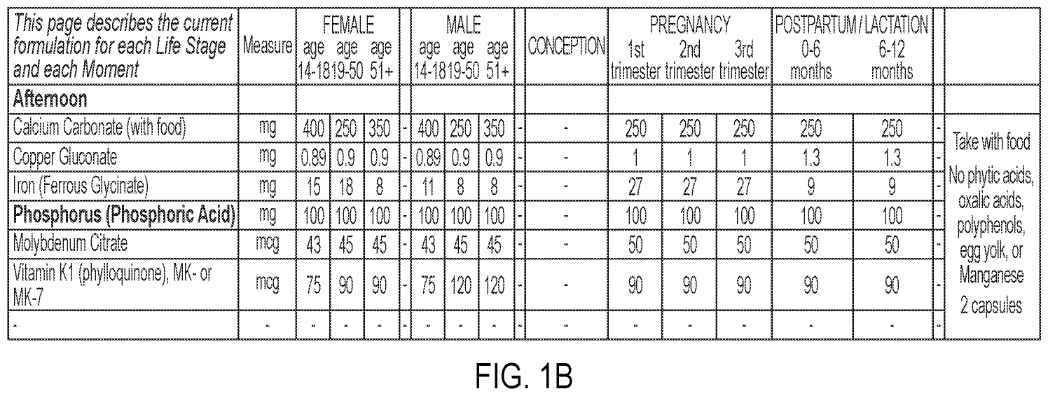

FIG. 1C is an exemplary table of evening components.

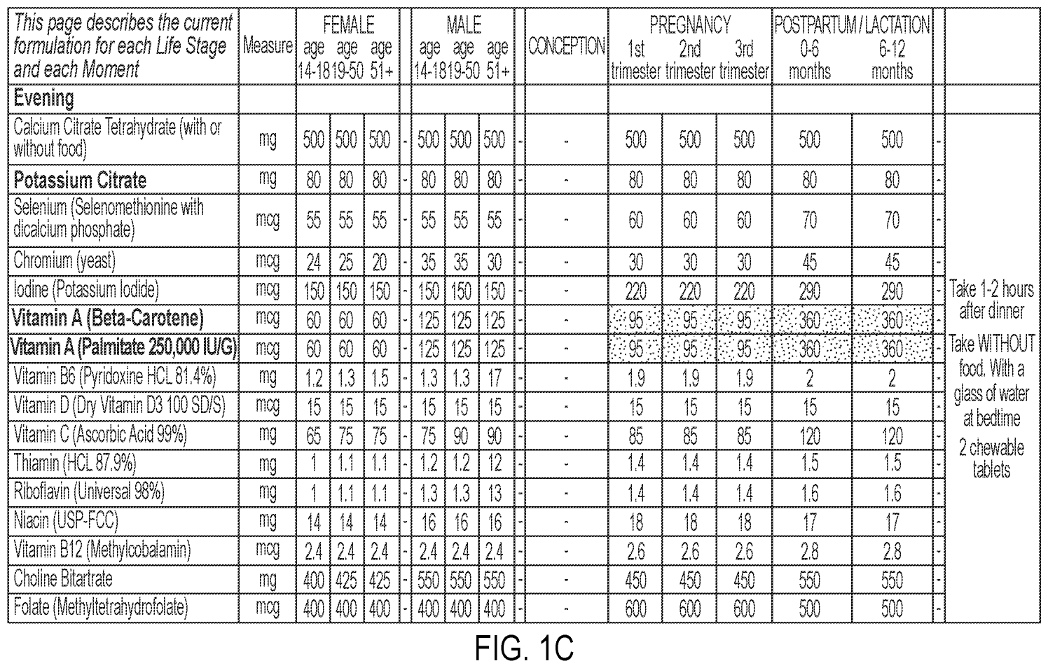

FIG. 1D is an additional exemplary table of evening components.

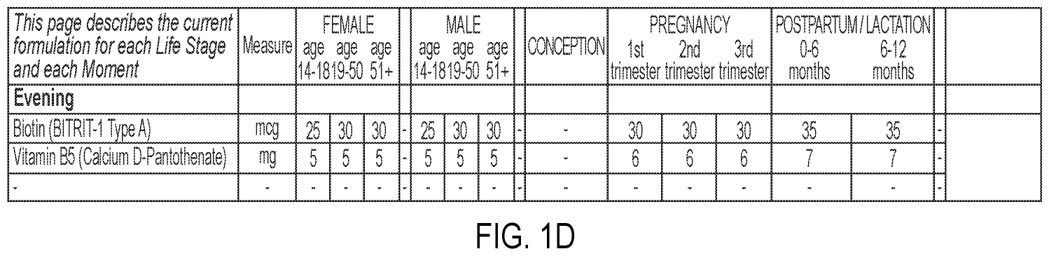

### DETAILED DESCRIPTION OF THE INVENTION

Dietary supplements or nutraceuticals provided in three-a-day permutations and tailored amounts based on an individual's age and gender. The three-a-day vitamin and mineral program is designed to provide optimum nutritional supplementation and absorption of what we have determined are the 26 most essential vitamins and minerals over the period of a 14 to 16-hour waking day, while mitigating compromised absorption due to food, drink and interactions with other nutrients in the program. Such permutations of each supplements or nutraceuticals being comprised of a combination of immediate and slow release components, dependent on improved pharmacodynamics and kinetics. In preferred embodiments, the amount of each nutrient never exceeds the Recommended Daily Allowance, so as not to exceed the body's absorbable limits and, in so doing, waste the excessive nutrients. Form factor and amount of such individual permutation of vitamins or minerals comprising the formulation may also be based on: biological sex and age of consumer; menstrational considerations, absorption and bioavailability of individual and collective mineral and vitamins; individual eating habits in consideration the chronology and contents of individual eating occasions (such as caffeine in the morning, three meals a day, and alcohol in the evening); antagonistic and synergetic properties of individual nutrients relative to other components of composition; Recommended Daily Allowance, Adequate Intake and toxicity quantities; positive and negative interaction of individual and collective nutrients of the composition with food in specific individual's diet. Of particular interest are one or more of the following vitamins and minerals: Calcium, spread among all three occasions in multiple forms and typically omitted from most nutraceutical supplementation programs; Magnesium, provided in a safe amount and in multiple forms in a time-released capsule to prevent inhibitory effects caused by the diet; Zinc, when consumed in excess in a supplement, can inhibit other nutrients' absorption; Iron, which has many contraindications with other nutrients and is separated from those inhibitors; and Vitamin A, where we have taken the reported average daily dietary intake of vitamin A into strong consideration when determining the amount of both carotenoid and retinoid amounts of vitamin A to prevent toxicity levels in all consumer types (pregnant, smokers, etc.). from traditional sleep aids. In some embodiments the components can be put into a single pill with timed release of the relaxant and stimulant or with the coating of the pill forming the initial drug release profile and the inner portion of the pill forming the latter drug release profile.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### 2. Formulations

Provided here are exemplary formulation embodiments provided in three-a-day permutations and tailored amounts based on an individual's age and gender. The formulation may be an oral formulation, and may comprise multiple pills or a single pill.

a. Morning Formulation

The morning formulation may comprise magnesium citrate, magnesium oxide, calcium carbonate, zinc carbonate, vitamin E (D-alpha tocopheryl acetate), and magnesium bisglycinate chelate.

b. Afternoon Formulation

The afternoon formulation may comprise calcium carbonate (with food), copper gluconate, iron (ferrous glycinate), phosphorus (phosphoric acid), molybdenum citrate, vitamin K1 (phylloquinone), MK-4 or MK-7.

c. Evening Formulation

The evening formulation may comprise calcium citrate tetrahydrate (with or without food), potassium citrate, selenium (selenometionine with dicalcium phosphate), chromium (yeast), iodine (potassium iodide), vitamin A (beta-carotene), vitamin A (palmitate 250,000 IU/G), vitamin B6 (pyridoxine HCl 81.4%), vitamin D (dry vitamin D3 100 SD/S), vitamin C (ascorbic acid 99%), thiamin (HCl 87.9%), riboflavin (universal 98%), niacin (USP-FCC), vitamin B12 (methylcobalamin), choline bitartrate, folate (methyltetrahydrofolate), biotin (BITRIT-1 Type A), Vitamin B5 (Calcium D-Pantothenate).

d. Pill Formation

The inner portion may comprise a capsule, and the outer portion may comprise a capsule coating. The capsule may be a softgel or a hard-shell capsule. The capsule may comprise a liquid fill, a powder fill, or a semi-solid fill; and a shell.

1. Liquid Fill

The liquid fill may comprise the stimulant, a liquid vehicle, a plasticizer, a surfactant, water, a solubilizing agent, and a suspending agent.

a. Liquid Vehicle

The liquid vehicle may comprise a lipophilic liquid or a semi-solid. The lipophilic liquid may comprise a vegetable oil and/or polyether. The semi-solid may comprise a hydrogenated oil, such as castor oil, and/or a wax such as bees wax.

b. Plasticizer

The plasticizer may comprise glycerol, glycerin, sorbitol, or propylene glycol, or a combination thereof.

c. Surfactant

The surfactant may comprise a lecithin, sorbitol, polysorbate, or sorbitan, or a combination thereof.

d. Solubilizing Agent

The solubilizing agent may comprise a beeswax or a mono-, di-, or triglyceride, or a combination thereof.

e. Suspending Agent

The suspending agent may comprise maltodextrin, sodium alginate, or xanthan gum, or a combination thereof.

2. Powder Fill

The powder fill may comprise the vitamins and minerals, a diluent, an anti-caking agent, and a lubricant.

a. Diluent

The diluent may comprise dicalcium phosphate, lactose, maltodextrin, microcrystalline cellulose, or a starch, or a combination thereof.

b. Anti-Caking Agent

The anti-caking agent may comprise magnesium silicate, silica gel, or talc, or a combination thereof.

c. Lubricant

The lubricant may comprise hydrogenated vegetable oil, magnesium stearate, mineral oil, or stearic acid, or a combination thereof.

3. Semi-Solid Fill

The semi-solid fill may comprise the vitamins and minerals, a semi-solid vehicle, a surfactant, and an emulsifying agent.

a. Semi-Solid Vehicle

The semi-solid vehicle may comprise hydrogenated palm oil, hydrogenated castor oil, cetyl alcohol, cetosteryl alcohol, a stearoyl polyoxylglyceride, a laurolyl polyoxyglyceride, or a combination thereof.

b. Surfactant

The surfactant may be a lecithin, sorbitol, polysorbate, or sorbitan, or a combination thereof.

c. Emulsifying Agent

The emulsifying agent may be polyethylene glycol, or poloxamer, or a combination thereof.

4. Shell

The shell may comprise a gelling agent, the plasticizer, an opacifier, a colorant, and water.

a. Gelling Agent

The gelling agent may comprise gelatin, a plant polysaccharide, a carrageenan, a modified starch, a cellulose or derivative thereof, or a combination of the foregoing. The modified starch may comprise starch hydrolysate. The cellulose derivative may comprise hypromellose or methylcellulose, or a combination thereof.

b. Opacifier

The opacifier may comprise titanium dioxide.

c. Plasticizer

The plasticizer may comprise glycerol, glycerin, sorbitol, or propylene glycol, or a combination thereof.

d. Coating

The coating may comprise a cellulose or derivative thereof, a polyether, a mineral oil, water, a plant resin or protein, and a surfactant. The polyether may comprise a polyethylene glycol, which may comprise a low molecular weight polyethylene glycol such as a PEG 300-600 or PEG 400, or a high molecular weight polyethylene glycol such as a PEG 4000-10,000, or a combination thereof.

Certain formulation embodiments may comprise the ingredients listed in Table 1.

Furthermore, although elements of the described aspects and/or embodiments may be described or claimed in the singular, the plural is contemplated unless limitation to the singular is explicitly stated. Additionally, all or a portion of any embodiment may be utilized with all or a portion of any other embodiment, unless stated otherwise.

While certain exemplary embodiments have been described and shown in the accompanying specification, it is to be understood that such embodiments are merely illustrative of and not restrictive on the broad invention, and that this invention is not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

1. A method consisting of taking an oral formulation supplement comprising three or more elements from the group consisting of: magnesium citrate, magnesium oxide, calcium carbonate, zinc citrate, vitamin E (D-Alpha tocopheryl acetate), and manganese bisglycinate chelate, taken in the morning.

2. A method consisting of taking an oral formulation supplement comprising three or more elements from the group consisting of: calcium carbonate (with food), copper gluconate, iron (ferrous glycinate), phosphorus (phosphoric acid), molybdenum citrate, vitamin K1 (phylloquinone), MK-4 or MK-7, taken in the afternoon.

3. A method consisting of taking an oral formulation supplement comprising three or more elements from the group consisting of: calcium citrate tetrahydrate (with or without food), potassium citrate, selenium (Selenomethionine with dicalcium phosphate), chromium (yeast), iodine (potassium iodide), vitamin A (beta-carotene), vitamin A (palmitate 250,000 IU/G), vitamin B6 (pyridoxine HCl 81.4%), vitamin D (dry vitamin D3 100 SD/S), vitamin C, (ascorbic acid 99%), thiamin (HCl 87.9%), riboflavin (universal 98%), niacin (USP-FCC), vitamin B12 (methylcobalamin), choline bitartrate, folate (methyltetrahydrofolate), biotin (BITRIT-1 Type A), vitamin B5 (calcium D-pantothenate), taken in the evening.

4. A method consisting of taking an oral formulation supplement of claim 1, wherein, the supplement comprises four or more from the group consisting of: magnesium citrate, magnesium oxide, calcium carbonate, zinc citrate, vitamin E (D-Alpha tocopheryl acetate), and manganese bisglycinate chelate, taken in the morning.

5. A method consisting of taking an oral formulation supplement of claim 2, wherein, the supplement comprises four or more from the group consisting of: calcium carbonate (with food), copper gluconate, iron (ferrous glycinate), phosphorus (phosphoric acid), molybdenum citrate, vitamin K1 (phylloquinone), MK-4 or MK-7, taken in the afternoon.

6. A method consisting of taking an oral formulation supplement of claim 3, wherein, the supplement comprises four or more from the group consisting of: calcium citrate tetrahydrate (with or without food), potassium citrate, selenium (Selenomethionine with dicalcium phosphate), chromium (yeast), iodine (potassium iodide), vitamin A (beta-carotene), vitamin A (palmitate 250,000 IU/G), vitamin B6 (pyridoxine HCl 81.4%), vitamin D (dry vitamin D3 100 SD/S), vitamin C, (ascorbic acid 99%), thiamin (HCl 87.9%), riboflavin (universal 98%), niacin (USP-FCC), vitamin B12 (methylcobalamin), choline bitartrate, folate (methyltetrahydrofolate), biotin (BITRIT-1 Type A), vitamin B5 (calcium D-pantothenate), taken in the evening.

7. A method consisting of taking an oral formulation supplement of claim 1, wherein, the supplement comprises five or more from the group consisting of: magnesium citrate, magnesium oxide, calcium carbonate, zinc citrate, vitamin E (D-Alpha tocopheryl acetate), and manganese bisglycinate chelate, taken in the morning.

8. A method consisting of taking an oral formulation supplement of claim 2, wherein, the supplement comprises five or more from the group consisting of: calcium carbonate (with food), copper gluconate, iron (ferrous glycinate), phosphorus (phosphoric acid), molybdenum citrate, vitamin K1 (phylloquinone), MK-4 or MK-7, taken in the afternoon.

9. A method consisting of taking an oral formulation supplement of claim 3, wherein, the supplement comprises five or more from the group consisting of: calcium citrate tetrahydrate (with or without food), potassium citrate, selenium (Selenomethionine with dicalcium phosphate), chromium (yeast), iodine (potassium iodide), vitamin A (beta-carotene), vitamin A (palmitate 250,000 IU/G), vitamin B6 (pyridoxine HCl 81.4%), vitamin D (dry vitamin D3 100 SD/S), vitamin C, (ascorbic acid 99%), thiamin (HCl 87.9%), riboflavin (universal 98%), niacin (USP-FCC), vitamin B12 (methylcobalamin), choline bitartrate, folate (methyltetrahydrofolate), biotin (BITRIT-1 Type A), vitamin B5 (calcium D-pantothenate), taken in the evening.

10. The method of claim 1 comprising five or more of group one, five or more of group two, and five of more of group three.

11. The method of claim 1 consisting essential of five or more from group one, five or more from group two, and five or more from group three.

12. The method of claim 11, tailored for an individual over 65 years of age.

13. The method of claim 1 taken with or just after a meal.

14. The method of claim 2 taken with or just after a meal.

15. The method of claim 3 taken with or just after a meal.
